(19) United States
(12) Patent Application Publication
(10) Pub. No.: US 2007/0088281 A1
(43) Pub. Date: Apr. 19, 2007

(54) Cover for protecting intravenous entry site

(76) Inventor: Connie Hopkins Ritchey, Lexington, KY (US)

Correspondence Address: Stockwell & Smedley, PSC, Suite 200, 861 Corporate Drive, Lexington, KY, 40503, US

(21) Appl. No.: 11/545,301

(22) Filed: Oct. 9, 2006

Related U.S. Application Data
(60) Provisional application No. 60/726,381, filed on Oct. 13, 2005.

(51) Int. Cl. A61M 5/32

(52) U.S. Cl. 604/174

(57) ABSTRACT

A cover for preventing wetness or contamination of an intravenous site is disclosed. The cover is manufactured in a variety of sizes and can fit on any extremity of the human body, and is equipped with a double-row of elastic bands, as well as a ruffled overhang.

### CROSS-REFERENCE TO RELATED APPLICATIONS

This application claims priority to U.S. Provisional Application No. 60/726,381, which was filed on Oct. 13, 2005.

### FIELD OF THE INVENTION

This invention relates generally to intravenous (IV) sites, and more specifically to a cover which can assist in preventing such a site from getting wet or contaminated.

### BACKGROUND OF THE INVENTION

Withdrawing and re-inserting an intravenous needle is both time-consuming and expensive. However, patients must also be bathed, and/or showered, including potentially in the area of the IV site. At present, bathing a patient's wound area can necessitate withdrawing and re-inserting an intravenous needle.

Patients also frequently have intermittent infusions for IV medications and the nurse converts the IV infusion to a heparin lock or saline lock for intermittent infusion. Like the above, it is desired to avoid having to re-stick the heparin lock or saline lock. Consequently, a means for addressing these problems is desired.

### SUMMARY OF THE INVENTION

It is an object of the present invention to provide an easily-installable means for protecting an IV site. It is another object of the present invention to achieve the above in multiple sizes so that it may fit the various sizes of human extremities. These and other objects and advantages of the invention will become readily apparent as the following description is read in conjunction with the accompanying drawings.

### BRIEF DESCRIPTION OF THE DRAWINGS

FIG. 1A shows an IV site, while FIG. 1B shows a first embodiment of the present invention protecting that site; and

FIG. 2A shows a second IV site, and FIG. 2B shows a second embodiment of the present invention protecting that site.

### DETAILED DESCRIPTION OF THE PREFERRED EMBODIMENTS

Before explaining the disclosed embodiment of the present invention in detail it is to be understood that the invention is not limited in its application to the details of the particular arrangement shown, since the invention is capable of other embodiments. Also, the terminology used herein is for the purpose of description and not of limitation.

The present invention is directed toward a cover for protecting intravenous (IV) sites. It's expensive to restart an IV in terms of nurse time and also equipment. It can also be painful for the patient. Sometimes it takes multiple sticks to get it into a patient's arm or extremity and find the correct vein. Some hospitals have an intravenous starter kit that might use tape or gauze. However, these kits can be expensive. The entire kit includes a dressing which is sterilized and requires a nurse to wear non-latex gloves.

To continually reapply intravenous units requires a lot of nursing time. Also, if the injury or the wound, or the entry point of the IV is exposed, the sterility of the dressing may be at risk. This could lead to nosocomial infection, otherwise known as a hospital-acquired hospital-induced infection. Also, IV infections are much more serious than other types of infections, and can more quickly lead to septicemia. Sterile dressings can be either gauze, tegaderm, or other mechanisms.

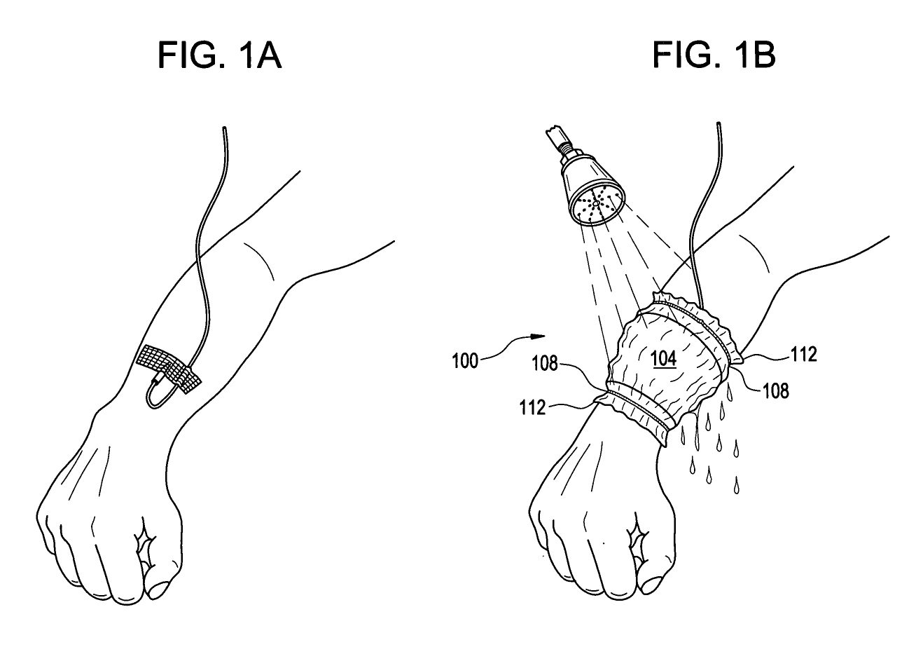

To address these and other concerns, FIG. 1B shows an IV site on an upper right arm of a patient with a sterile dressing such as one of those discussed above. FIG. 1B shows an exemplary embodiment of the cover 100 of the present invention positioned about the IV site of FIG. 1A.

From FIG. 1B it is apparent that that the cover 100 has a main body 104, as well as double-elastic bands 108 arranged in a double row, and also an ruffled overhang 112. As shown in FIG. 1B, the present invention wraps around the arm or other extremity of a wearer and can be cylindrical in shape with elastic or clinging bands 108 at either end thereof. The cylinder of the body 104 is open-ended and slides over an IV site.

The double-elastic bands 108 have the effect of increasing the sealability of the cover 100, while not applying undue pressure to the skin surrounding the site for which protection is needed. Slipping and movement of the cover 100 is also reduced because of the double-elastic band 108, as well as reducing water intrusion. The elastic bands 108 can be integrally molded into the cover 104, either on the inside, or on the outside.

Similarly, the ruffled overhang 112 provides additional protection for the site, yet does not restrict movement by the wearer, and also does not make the cover 100 intrusive or awkward to install.

The body 104 of the cover 100 can be made of the same type of plastic as shower caps, and is also waterproof. An exemplary embodiment could be made of low- or high-density polyethylene, although the present invention should not be considered as limited exclusively thereto.

In the embodiment of the cover 100 that is intended to be fitted on a human arm, the cover 100 can be manufactured so that an upper opening can be larger than a lower opening. This is useful because most people's arms are not of uniform size throughout their length. Consequently, the gripping surface of an upper opening must be a different size than the gripping surface of a lower opening.

It is important that the cover 100 be non-irritating to human skin. The double-row of elastic bands 108 are molded or embedded within the cover 100, to assist in protecting the IV site. However, the double-row of elastic bands 108 do not grip so tightly that they interfere with the I.V. tubing. Thus, some water seepage is possible, so that even while using the present invention, it would still be necessary for a nurse and patient to exercise care with the showerhead. Still, the water must travel through not one but two elastic bands.

The cover 100 is only meant to reasonably prevent water or other substances from reaching the IV site. It is possible that under enough pressure or volume of water from a showerhead or other cleansing mechanism, some water could seep into the IV site. In such a case, the cover 100 could be removed, discarded, and replaced.

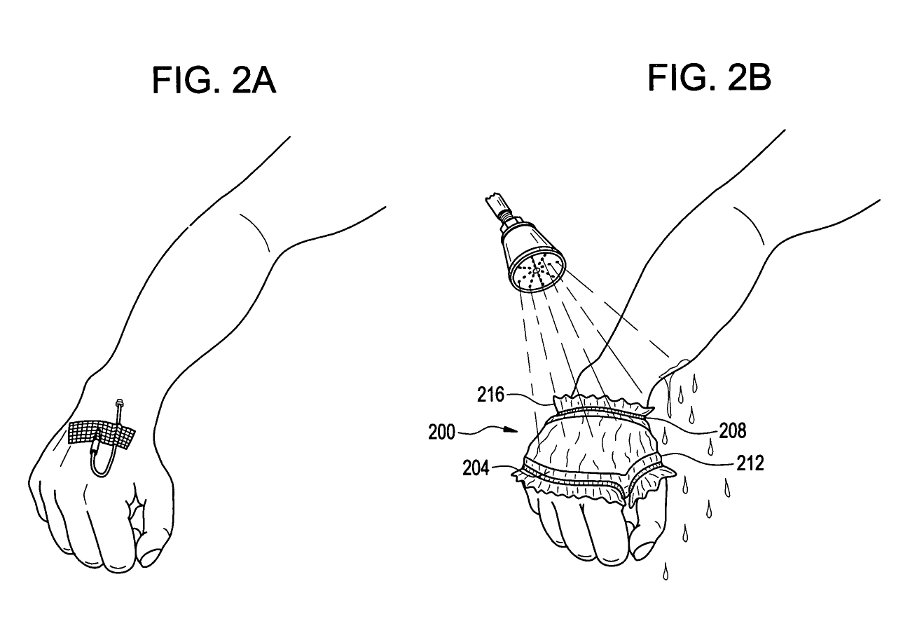

FIG. 2A shows another common location of an IV site, at the top of the right hand of a patient. FIG. 2B shows a second embodiment of the present invention, in which a cover 200 is not entirely cylindrical in shape, but has an extra hole for admitting a thumb so as to protect the IV site of FIG. 2A The cover 200 has a finger band 204, wrist band 208, and thumb band 212. All bands 204 208, and 212 can be composed of double-elastic rows, although this is mentioned for exemplary purposes only, so that the present invention should not be considered as limited exclusively thereto. The cover 200 also has an overhang 216, which serves the same purpose as the overhang 112.

Unlike the embodiment shown in FIGS. 1A and 1B, in the FIGS. 2A and 2B the IV tubing is shown being temporarily disconnected, although the IV site is still active. One example where this could occur is with a Heparin™ lock. Under such circumstances, water seepage would be less of a problem, and potentially could be avoided altogether. Although not shown, the IV tubing of FIG. 1A could also be disconnected as shown in FIG. 2A.

The present invention also contemplates other embodiments of the cover which could be adapted to fit other extremities where application of an IV is also suitable. One example of such an area is the upper leg. Additionally, the present invention could also be applied to peripheral intercannula catheters (PICs). Furthermore, the present invention could also be applied to other types of non-IV sites that need protection, such as stitches or sutures.

The body 104 of the cover 100 can also be made of nitrile latex, although other compositions are contemplated within the spirit and scope of the present invention. Other substances could include a durable semi-permeable membrane in which the molecules are 3 or 4 nanometers apart, such as Gore-Tex™. Another material may be polyurethane.

Such a product could be manufactured by positioning a membrane gel, such as but not limited to a latex membrane, between slices of a loose-weave nylon formation layer and inserted in a baking or ceramic oven. The resulting combination of gel and nylon could then be heated to a specific temperature such as 300 F, with certain areas embedded with a heat-sensitive stripe of sealant that will bind all materials touching the sealant into a type of seam. However, the present invention should not be considered as limited thereto.

After returning to room temperature, the product could be chopped into grids, with the sealant-areas providing what will eventually be the seams of the product. The chopping process could also include perforations which would be useful for a tear-off packaging implementation, but could also be completely separated by some other type of cutting process. The printed chopped sheets could then be gamma-radiated for sterilization purposes, because gamma rays have proven to be effective at killing existing bacteria, and discouraging their further growth. It is not required that the cover be manufactured with seams. Instead, it could also be fabricated with a high-stress elasticizing process.

The cover 100 could be manufactured like a trash-bag that comes printed flat on a roll, where the user then tears off a single unit, and then fingers it open, before inserting the extremity. The cover 100 could also come in a disposable, sanitary single use package. In the embodiment where the upper and lower openings of the cover 100 vary in size (to accommodate the non-uniformity of a human arm), the roll of covers 100 could be manufactured in a large-to-large, small-to-small, large to large capacity. In other words, on a roll of a substantial number of covers 100, a larger and of a cover could be printed next to another larger end, and then a smaller end opposite that larger end could be printed next to another smaller end. Doing so could reduce the amount of un-needed material used in manufacturing the present invention.

It is anticipated that various changes may be made in the arrangement and operation of the system of the present invention without departing from the spirit and scope of the invention, as defined by the following claims.

What is claimed is:

1. A cover to be applied over an intravenous site, comprising:
a hollow cylindrical body, having a transparent appearance and being composed of a flexible waterproof material;
upper and lower double-row elastic bands incorporated within said body; and
a ruffled overhang, positioned at the outer edge of the elastic bands.

2. The cover of claim 1, wherein said body is made from low- or high-density polyethylene material.

3. The cover of claim 1, wherein said body is waterproof.

4. The cover of claim 1, wherein said body is non-irritating to human skin.

5. The cover of claim 1, wherein said elastic bands grip sufficiently to hold their position but not interfere with the I.V. tubing.

6. The cover of claim 1, wherein said wraps is positioned around the arm or other extremity of a wearer.

7. The cover of claim 1, wherein said cylinder of said body is open-ended and slides over an intravenous site.

8. The cover of claim 1, wherein said cover is not entirely cylindrical, but also has hole for admitting a thumb.

9. The cover of claim 8, wherein said cover has a finger band, wrist band, and thumb band, each equipped with a double-row elastic band.

10. The cover of claim 1, wherein said cover is packaged within a disposable, sanitary single use package.

11. The cover of claim 1, wherein said cover is used to protect stitches or sutures instead of intravenous sites.

12. The cover of claim 1, wherein said body is made of nitrile latex.

13. The cover of claim 1, wherein said body is made of a durable semi-permeable membrane in which the molecules are 3 or 4 nanometers apart.

14. The cover of claim 1, wherein said body is made of Gore-Tex™.

15. The cover of claim 1, wherein said manufactured like a trash-bag that comes printed flat on a roll, where the user then tears off a single unit, and then fingers it open, before inserting the extremity.

16. The cover of claim 1, wherein said body manufactured by positioning a membrane gel, such as but not limited to a latex membrane, between slices of a loose-weave nylon formation layer and inserted in a baking or ceramic oven.

17. The cover of claim 16, wherein the above combination is heated to a specific predetermined temperature.

18. The cover of claim 17, wherein the above combination said specific predetermined temperature is 300 F.

19. The cover of claim 16, wherein the above combination further comprises:
pre-designated areas embedded with a heat-sensitive stripe of sealant that will bind all materials touching the sealant into a type of seam.

20. A method of manufacturing a cover for protecting an intravenous site on a human extremity, comprising:
positioning a membrane gel such as but not limited to a latex membrane;
between slices of a loose-weave nylon formation layer;
inserted said combination in a baking or ceramic oven incorporating upper and lower bands in a double-row arrangement; and
heating said combination to a predetermined temperature.

21. The method of claim 20, wherein said specific predetermined temperature is 300 F.

22. The method of claim 20, further comprising:
applying sealant at pre-determined locations within said combination to produce seams and/or folds.

23. The method of claim 20, further comprising:
returning said combination to room temperature.

24. The method of claim 23, further comprising:
chopping said combination into grids, wherein sealant-areas resulting in seams.

25. The method of claim 24, wherein said chopping step further comprises:
forming perforations which would be useful for a tear-off spooled packaging mode.

26. The method of claim 14, further comprising:
gamma-radiating said combination, for sterilization purposes.

27. The method of claim 14, further comprising:
high-stress elasticizing process.

28. A method of protecting an intravenous site on a human extremity, comprising:
removing a hollow site cover from a sanitary enclosure;
sliding said cover along said extremity until it covers said site; and
tightening upper and lower bands incorporated within said cover until they fit snugly upon said extremity.
